(19) United States
(12) Patent Application Publication
(10) Pub. No.: US 2010/0099631 A1
(43) Pub. Date: Apr. 22, 2010

(54) Novel Use

(76) Inventor: John Alan Hawley, Victoria (AU)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/439,245

(22) PCT Filed: Jul. 6, 2007
(86) PCT No.: PCT/EP07/56897

§ 371 Date: Oct. 22, 2009

(30) Foreign Application Priority Data
Aug. 31, 2006 (GB) 0617182.1

(51) Int. Cl. A61K 31/70; A61K 31/522

(52) U.S. Cl. 514/23; 514/263.34

(57) ABSTRACT

The present invention relates to the use of the combined oral administration of caffeine with carbohydrate for increasing the rate of muscle glycogen resynthesis after strenuous exercise.

The present invention relates to the use of the combined oral administration of caffeine with carbohydrate for increasing the rate of muscle glycogen resynthesis after strenuous exercise.

Endogenous carbohydrate in the form of muscle glycogen is the primary fuel source during both prolonged continuous moderate-intensity exercise, (longer than 90 minutes), and intense intermittent exercise typical of the pattern of many team sports, (McInerney P, Lessard S. J., Burke L. M., Coffey V. G., Lo Giudice S. L., Southgate R. J., Hawley J. A., Failure to repeatedly supercompensate muscle glycogen stores in highly trained men. Med Sci Sports Exerc. 37:404-411, 2005). Therefore, a major goal for individuals involved in these activities is to achieve high muscle glycogen levels prior to the start of exercise.

Restoration of muscle glycogen stores after exercise is crucial for the recovery of subsequent exercise capacity. When adequate carbohydrate is ingested following strenuous activity (i.e. 10 grams of carbohydrate per kilogram of body mass per day), muscle glycogen restoration can be attained within 24-36 hours. However, nutritional strategies to rapidly restore muscle glycogen within a short-time period (e.g. <12 hours) are not well defined. It is reported that for athletes involved in sports that involve multiple exercise bouts within a short time frame, it would be beneficial to identify nutritional guidelines that maximise the rate of muscle glycogen storage in the early hours post-exercise (Jentjens R., Jeukendrup A. Determinants of post-exercise glycogen synthesis during short-term recovery. Sports Med. 33:117-144, 2003).

Caffeine has been used as an ergogenic aid in numerous sporting situations (Graham T. E. Caffeine and Exercise: metabolism, endhurance and performance. Sports Med. 31:785-807, 2001). More recently a study by Yeo et al, (Yeo S E, Jentjens R L, Wallis G A, Jeukendrup A E. Caffeine increases exogenous carbohydrate oxidation during exercise. J Appl Physiol 99:844-50), showed that the co-ingestion of caffeine and glucose during exercise leads to an increase in muscle glucose oxidation by 26%, while other researchers have found that caffeine plays a role in altering substrate selection by muscle (Graham T. E. Caffeine and Exercise: metabolism, endhurance and performance. Sports Me. 31:785-807, 2001). All these papers have shown that caffeine can enhance exercise performance and has a potent role in altering fuel metabolism.

Unexpectedly, the present inventors have now found that caffeine when administered with carbohydrate is capable of enhancing the restoration of muscle energy stores by increasing the rate of glycogen resynthesis after exercise, when compared to administration of carbohydrate alone.

Accordingly there is provided the use of caffeine and carbohydrate in the manufacture of a nutritional composition for oral administration after exercise for increasing the rate of muscle glycogen resysnthesis.

Suitable sources of caffeine (methylxanthine) include both synthetically manufactured caffeine and caffeine occurring naturally in products such as coffee, tea, cacao, cola nut, gurana, yerbamate, and other naturally occurring plant sources and mixtures thereof.

Caffeine for use in the present invention is suitably synthetic and more suitably in the form of anhydrous caffeine.

Suitably nutritional compositions for use in the present invention comprise from 0.001 to 0.5% w/w caffeine.

Suitable sources of carbohydrate include but are not limited to glucose, glucose syrup, glucose-fructose syrup, sucrose, maltose, lactose, fructose, maltodextrins, starches, oligosaccharides, and other polysaccharides and mixtures thereof.

Suitably nutritional compositions for use in the present invention comprise from 1 to 90% w/w carbohydrate.

A nutritional composition for use in the present invention may be in the form of a beverage, particularly a beverage that is ready to drink with from 0.001 to 0.5% w/w caffeine and from 1 to 40% w/w carbohydrate. More suitably the composition is in the form of a ready to drink beverage with from 0.01 to 0.2% w/w caffeine and from 2 to 25% w/w of carbohydrate. Beverages may be still or carbonated.

Beverage compositions for use in the present invention may also be in the form of a solid or a liquid concentrate for dilution with a liquid for the preparation of a beverage that is ready to drink.

A solid-concentrate composition may be in the form of a powder for re-constitution with a liquid, typically water, prior to ingestion. A powder composition may comprise from 0.005 to 0.5 w/w caffeine and from 1 to 90% w/w carbohydrate in the concentrate. For example 39 g of a powder composition when reconstituted in 500 ml of water may comprise from 0.001 to 0.2% w/w caffeine and 2 to 25% w/w carbohydrate.

Nutritional compositions for use in the present invention may also be in the form of an edible solid such as a tablet or a nutritional bar or in the form of a semisolid such as a gel.

A tablet composition may be dissolved or dispersed in water prior to consumption or may be ingested directly without being dissolved or dispersed in water. For example, a 3.5 g tablet may comprise from 0.001 to 0.2% w/w caffeine and 10 to 90% w/w carbohydrate.

A nutritional bar may be a cereal-based composition intended to provide energy. For example a 50 g nutritional bar composition may comprise from 0.001 to 0.5% w/w caffeine and from 10 to 80% w/w carbohydrate.

A gel composition may be prepared as a single dose for consumption and may suitably be followed by consumption of a liquid, typically water. Alternatively, a gel composition may be dissolved or dispered in water prior to consumption. For example a 45 g gel composition may comprise from 0.001 to 0.5% w/w caffeine and from 10 to 80% w/w carbohydrate.

An advantage of compositions for use in the present invention is that the rate of muscle glycogen resysnthesis may be increased by up to 66% when compared to ingesting carbohydrate alone after a bout of glycogen-depleting exercise is performed.

In a further aspect, the present invention provides a method of promoting muscle glycogen resynthesis following a bout of glycogen-depleting exercise, which method comprises ingesting a nutritional composition comprising caffeine and carbohydrate.

Compositions for use in the present invention may further comprise ingredients commonly used in the field of nutritional compositions.

The present invention is illustrated by way of the following non-limiting examples.

Methods

Eight trained cyclists participated in a study which was approved by the Ethics Committee of RMIT University, Melbourne, Australia. Each subject participated in two experimental trials separated by 7 to 10 days. Trials were randomised and double-blind. Approximately 12 to 14 hours before each trial, the subjects reported to the laboratory to undertake 90 minutes of intense cycling (repeated sprints) to deplete muscle glycogen stores. The subjects were then fed a standardised low carbohydrate meal (60% of energy from fat) and had to refrain from solid feeding for the following 12 to 14 hours. During this period water was allowed ad libitum.

The next morning, subjects reported to the laboratory between 0600 and 0700 hours. After a rest period of 10 minutes, an indwelling canula was inserted into the right forearm and a resting blood sample taken. Local anaesthesia was applied to the subjects skin to enable subcutaneous tissue and fascia of the vastus lateralis of the subjects right leg in preparation for muscle biopsies.

After biopsy preparation, a bout of exhaustive exercise (submaximal continuous cycling) was undertaken to further deplete muscle glycogen stores. The exhaustive cycling protocol has been previously described by McInerney et al. (McInerney P., Lessard S. J., Burke L. M., Coffey V. G., Lo Giudice S. L., Southgate R. J., Hawley J. A. Failure to repeatedly supercompensate muscle glycogen stores in highly trained men. Med. Sci. Sports Excer. 37:404-411, 2005). During exercise the subjects were allowed to drink water ad libitum and were fan-cooled.

Laboratory conditions were standardised for the tests with 50% relative humidity and a temperature of 20° C. Immediately on completion of the exercise and while subjects remained seated on the cycle ergometer, a muscle biopsy was taken and frozen within 15 seconds of the last muscle contraction. After the biopsy, subjects dismounted the ergometer and rested in a supine position. During one trial, subjects were fed 1 g/kg body mass (BM) of carbohydrate immediately upon cessation of exercise and thereafter 1 g/kg BM of carbohydrate after 60, 120 and 180 minutes of recovery (a total of 4 g/kg BM carbohydrate). In the second trial, subjects followed the same carbohydrate ingestion regimen but in addition consumed 4 mg/kg BM of caffeine immediately upon cessation of the exercise and then after 120 minutes during recovery. Blood samples were taken at regular intervals throughout the recovery period (0, 30, 60, 90, 120, 180 and 240 minutes). Muscle biopsies were taken immediately after exercise and after 1 and 4 hr of recovery. All muscle samples were stored at −80° C. until analysis.

Analysis

Blood samples were analysed for plasma glucose and insulin concentrations at rest, and at regular intervals during recovery. The protocols for blood analysis are routine and have been described previously ((McInerney P., Lessard S. J., Burke L. M., Coffey V. G., Lo Giudice S. L., Southgate R. J., Hawley J. A. Failure to repeatedly supercompensate muscle glycogen stores in highly trained men. Med. Sci. Sports Excer. 37:404-411, 2005). Plasma caffeine levels were analysed by high-performance liquid chromatography. Muscle samples were analysed for glycogen content immediately after exercise and after 1 and 4 hours of recovery.

Results

Blood Glucose and Insulin Concentrations

Blood glucose and insulin concentrations are displayed in Table 1 and FIG. 1. There were no significant differences for either blood glucose or insulin levels at rest and immediately post exercise. As would be expected, blood glucose levels rose significantly within 30 min of carbohydrate ingestion at the cessation of exercise in both trials (P<0.05). However, the ingestion of caffeine with carbohydrate resulted in a significantly greater area under the insulin versus time curve compared to when carbohydrate alone was ingested (P<0.05).

TABLE 1
Plasma Glucose and Insulin Concentration 4 Hours post exercise.
Plasma Glucose and Plasma Insulin Concentration

| | | |
|---|---|---|
| | Glucose (mmol/L) | Insulin (uU/mL) |
| Time | Treatment Group | Treatment Group |

| (hr) | Placebo | Caffeine | Placebo | Caffeine |
|---|---|---|---|---|
| rest | 3.93 | 4.04 | 8.30 | 9.36 |
| 0 | 3.49 | 3.62 | 4.26 | 4.17 |
| 0.5 | 6.05 | 6.06 | 24.35 | 30.17 |
| 1 | 5.94 | 6.29 | 24.49 | 29.23 |
| 1.5 | 5.35 | 5.95 | 34.97 | 46.33 |
| 2 | 5.70 | 5.58 | 34.00 | 46.02 |
| 3 | 4.70 | 5.24 | 44.28 | 68.53 |
| 4 | 4.56 | 5.23 | 36.78 | 46.91 |

Plasma Caffeine Concentrations

Plasma caffeine concentrations are displayed in Table 2 and FIG. 2. All subjects refrained from caffeine ingestion before a trial, as confirmed by the absence of caffeine in resting blood sample. As intended, carbohydrate and caffeine resulted in a significant increase in plasma caffeine levels such that after 1 hr values had risen to 30 umol/L and after 4 hr had climbed to ˜80 umol/L (P<0.001).

TABLE 2
Plasma Caffeine Concentration 4 hours post exercise.
Plasma caffeine concentration (uM)

Treatment Group

| Time (hr) | Placebo | Caffeine |
|---|---|---|
| rest | 0.00 | 0.00 |
| 0 | 0.00 | 0.00 |
| 1 | 0.00 | 31.52 |
| 4 | 0.00 | 77.86 |

(Since the placebo does not contain caffeine, there is no increase in the plasma caffeine concentration in FIG. 2, hence no chart is seen for the placebo).

Muscle Glycogen

At exhaustion, muscle glycogen levels were ˜80 mmol·kg⁻¹ d.w, with no significant differences observed between the two trials (74±21 vs. 76±9 mmol/kg) for placebo and caffeine respectively. After 1 hr of recovery, muscle glycogen content was increased by a similar amount (˜80%) in both trials (121±9 vs. 149±18 mmol/kg d.w for placebo (PL) and caffeine (CAFF) respectively. However, after 4 hr of recovery the co-ingestion of caffeine with CHO resulted in significantly higher glycogen levels (313±26 vs. 234±20 mmol/kg d.w., P<0.001). Accordingly, the rates of muscle glycogen synthesis from 1-4 hr were significantly higher (66%) in CAFF than PL (57.7±7.6 vs. 38.0±3.2 mmol/kg/hr; P<0.05), (Table 3 and FIG. 3).

Accordingly, the average rate of resynthesis over the 4 hours of recovery was significantly higher with CAFF compared to PL (57.71±7.6 vs. 38.02±3.2 mmol/kg/hr; P<0.05; 66%), Table 4 and FIG. 4).

TABLE 3
Muscle Glycogen Content 4 hours post exercise.
Muscle Glycogen Content (mmol/Kg · dm)

Treatment Group

| Time (hr) | Placebo | Caffeine |
|---|---|---|
| 0 | 74 | 76 |
| 1 | 121 | 149 |
| 4 | 234 | 313 |

TABLE 4
Muscle Glycogen Resynthesis Rate post exercise.
Muscle Glycogen Resynthesis Rate (mm/Kg · hr · dm)

Treatment Group

| Time | Placebo | Caffeine |
|---|---|---|
| 0-1 hr | 65.19 | 63.52 |
| 1 hr-4 hr | 29.70 | 57.69 |

Conclusion.

The results from the present study demonstrate that the co-ingestion of caffeine with carbohydrate results in significantly greater rates of muscle glycogen resynthesis than when carbohydrate alone is ingested. These findings are novel in the field of muscle metabolism and applied nutrition.

### EXAMPLE 1

TABLE 5
Sport Drink Formulation - 2% w/w Carbohydrate,
0.01% w/w Caffeine.

| Ingredient | g/l | % w/w |
|---|---|---|
| Liquid carbohydrate blend, | 28.41 | 2.818 |
| approx 70% w/w solids | | |
| Caffeine | 0.1 | 0.01 |
| Citric Acid | 4.66 | 0.462 |
| Acididty regulator | 2.02 | 0.200 |
| Preservative | 0.37 | 0.0370 |
| Sweetener | 0.213 | 0.0213 |
| Ascorbic Acid | 0.24 | 0.024 |
| Gum | 0.36 | 0.036 |
| Cloudifier | 0.4 | 0.040 |
| Flavour | 0.16 | 0.016 |
| Colour | 0.004 | 0.0004 |
| Water | To 11 | to 100% |

### EXAMPLE 2

TABLE 6
Sport Drink Formulation - 8% w/w Carbohydrate, 0.1% w/w Caffeine.

| Ingredient | g/l | % w/w |
|---|---|---|
| Liquid carbohydrate blend, | 117.5 | 11.374 |
| approx 70% w/w solids | | |
| Caffeine | 1.03 | 0.100 |
| Citric Acid | 4.66 | 0.451 |
| Acididty regulator | 2.02 | 0.196 |
| Preservative | 0.37 | 0.0370 |
| Sweetener | 0.213 | 0.0213 |
| Ascorbic Acid | 0.24 | 0.0234 |
| Gum | 0.36 | 0.035 |
| Cloudifier | 0.4 | 0.040 |
| Flavour | 0.16 | 0.016 |
| Colour | 0.004 | 0.0004 |
| Water | To 11 | to 100% |

### EXAMPLE 3

TABLE 7
Sport Drink Formulation with 25% w/w carbohydrate,
0.2% w/w Caffeine

| Ingredient | g/l | % w/w |
|---|---|---|
| Liquid carbohydrate blend | 392.0 | 35.382 |
| approx 70% w/w solids | | |
| Caffeine | 2.20 | 0.200 |
| Citric Acid | 4.66 | 0.424 |
| Acididty regulator | 2.02 | 0.183 |
| Preservative | 0.37 | 0.034 |
| Ascorbic Acid | 0.24 | 0.022 |
| Gum | 0.36 | 0.033 |
| Cloudifier | 0.40 | 0.036 |
| Flavour | 0.16 | 0.015 |
| Colour | 0.004 | 0.0004 |
| Water | To 11 | to 100% |

1. (canceled)

2. The method according to claim 11 wherein the caffeine is present in the nutritional composition in an amount 0.001 to 0.5% w/w.

3. The method according claim 2 wherein the source of caffeine is selected from synthetically manufactured caffeine and caffeine occurring naturally in coffee, tea, cacao, cola nut, gurana, yerbamate, and other naturally occurring plant sources and mixtures thereof.

4. The method according to claim 11 wherein the carbohydrate is present in an amount from 1 to 90% w/w.

5. The method according to claim 5 wherein the source of carbohydrate is selected from glucose, glucose syrup, glucose-fructose syrup, sucrose, maltose, lactose, fructose, maltodextrins, starches, oligosaccharides, and other polysaccharides and mixtures thereof.

6. The method according to claim 11 wherein the nutritional composition is a ready to drink beverage or a liquid or solid concentrate for the preparation of a ready to drink beverage.

7. The method according to claim 6 wherein the ready to drink beverage is a still drink, or a carbonated soft drink or a health drink.

8. The method according to claim 11 wherein the nutritional composition is in the form of a tablet.

9. The method according to claim 11 wherein the nutritional composition is in the form of gel.

10. The method according to claim 11 wherein the nutritional composition in the form of a nutritional bar.

11. A method of promoting muscle glycogen resynthesis after a glycogen-depleting exercise in a human in need thereof, which comprises administering to said human an effective amount of a nutritional composition comprising caffeine and carbohydrate.

12. The method according to claim 8 wherein the tablet may contain 0.001 to 2% w/w caffeine and 10 to 90% w/w carbohydrate.

13. The method according to claim 10 wherein the nutritional bar may contain 0.001 to 0.5% w/w caffeine and 10 to 80% w/w carbohydrate.

14. The method according to claim 9 wherein the nutritional gel may contain 0.001 to 0.5% w/w caffeine and 10 to 80% w/w carbohydrate.

15. The method according to claim 11 wherein the nutritional composition is a powder for reconstitution and contains from may contain 0.001 to 0.5% w/w caffeine and 1 to 90% w/w carbohydrate prior to reconstitution.
